(19) United States
(12) Patent Application Publication
(10) Pub. No.: US 2011/0117043 A1
(43) Pub. Date: May 19, 2011

(54) METHODS OF COATING EYELASHES AND EYELIDS WITH A LONG-WEARING MASCARA COMPOSITION

(76) Inventors: James Herman Dempsey, Danbury, CT (US); Thomas Elliot Rabe, Baltimore, MD (US)

(21) Appl. No.: 12/912,418

(22) Filed: Oct. 26, 2010

Related U.S. Application Data
(60) Provisional application No. 61/255,451, filed on Oct. 27, 2009.

(51) Int. Cl. A61Q 1/10; A61K 8/92; A45D 40/00

(52) U.S. Cl. 424/70.7; 132/209; 132/314

(57) ABSTRACT

A method of coating eyelashes and/or eyelids with a cosmetic composition that includes from about 15% to about 50%, by weight, of a film former selected from the group consisting of tall oil glyceride, pentaerythrityl rosinate, glyceryl rosinate, and the hydrogenated versions and mixtures thereof. Further there is from about 0.1% to about 14%, by weight, of a colorant. The cosmetic composition is long lasting and has excellent wear characteristics.

### CROSS REFERENCE TO RELATED APPLICATION

This application claims the benefit of provisional application No. 61/255,451, filed Oct. 27, 2009.

### FIELD OF THE INVENTION

This invention relates to long-wearing mascara compositions and methods of coating eyelashes, eyebrows, and/or eyelids with the same. The mascara composition may be formulated for use as an eyeliner or brow product. The mascara can last on the eyelashes and/or eyelids in excess of 24 hours and in certain embodiments in excess of 48 hours. The methods of the present invention can include applying mascara to the eyelashes as a single layer application or as a first layer with a top coat. A solvent-based removal composition is also disclosed because the mascara compositions of the present invention have very low solubility in soap and water.

### BACKGROUND OF THE INVENTION

Mascara compositions have been used for many years to increase the thickness, length and overall appearance of human eyelashes. There are generally two types of mascara: regular and water proof. Regular mascara is generally water soluble, while the water proof variety can be removed with soap and water. Both are typically applied only once per day. As the day progresses, the mascara gradually wears off. Current mascaras wear off relatively quickly and reapplication during the day is necessary but inconvenient. Moreover, current mascara is easily worn off by physical abrasion. If the small amount of mascara remaining on the eyelashes at night is not removed, it will gradually rub off during the night onto the sleeping surface leaving an undesirable mess. Women prefer to have the look that mascara provides at all times during the day. But the application process can be time consuming and inconvenient. Thus, women often sacrifice the look they desire because they simply do not have the time, or are not able to continually reapply mascara.

“Long Wear” mascaras are also available. But “long” is a relative term, and existing long wear mascaras are designed to eliminate reapplication during the day, and are generally not suitable for multi-day wear. One of the problems with long wear mascara is that making the composition sufficiently sticky to stay on the lashes, it is also sufficiently sticky to cause excessive clumping of the lashes. Some clumping is inevitable, but if too many individual lashes stick together and appear as large clumps, the consumer does not get the desired look.

Two layer mascaras are known, but the top coat is typically a visual agent. That is, the second coat compliments the first, or brings a second color. But these are typically not intended for wear beyond the normal 12-16 hour day. And these two layer mascaras are typically removed at night with soap and water.

There exists a need for methods of applying a mascara composition, either one or two layers, that can last overnight on a person's eyes without degradation and without wearing off on sheets, pillow cases and other bed clothes. There is a need for a method of applying a mascara composition that lasts more than 24 hours and achieves the desired look of current mascaras and the consumer desired feel. Further there is a need for a solvent-based mascara remover that can remove mascara compositions that are not generally soluble in soap and water. The methods and compositions of the present invention meet these and other needs.

### SUMMARY OF THE INVENTION

In one aspect of the present invention there is provided a method of coating eyelids and eyelashes comprising the step of applying to eyelashes, eyelids, or both, a mascara composition. The mascara composition comprising: from about 15% to about 35%, by weight, of a component selected from the group consisting of tall oil glyceride, pentaerythrityl rosinate, glyceryl rosinate, and the hydrogenated versions and mixtures thereof and from about 0.1% to about 14%, by weight, of a colorant.

Further, there is provided a method of coating eyelids and eyelashes comprising the steps of applying to eyelashes, eyelids, or both, a mascara composition comprising a two layer cosmetic composition. The cosmetic composition comprises a first and second layer. The first layer comprising from about 15% to about 35%, by weight, of a film former selected from the group consisting of tall oil glyceride, pentaerythrityl rosinate, glyceryl rosinate, and the hydrogenated versions and mixtures thereof; and from about 0.1% to about 14%, by weight, of a colorant. The second layer comprises a clear polymer selected from the group consisting of organosiloxane resins and mixtures thereof.

The methods and compositions of the present invention provide a mascara composition that lasts at least about 24 hours on consumers' eyelashes and/or eyelids without appreciable wear, preferably, the mascara lasts 36 hours without wearing off and even more preferably the mascara lasts 48 hours or longer without wearing off or cracking. The methods and compositions of this invention can be provided as a single layer or as a long-lasting base coat with a film-forming top coat that improves the longevity of the combined composition. Whether the mascara is provided as one layer or two, it cannot be generally soluble in soap and water. It is necessary for the mascara user to wash her face at night without removing the mascara. Given the low solubility of the mascara, or the mascara/top coat, in soap and water, a solvent-based remover is also provided.

### DETAILED DESCRIPTION OF THE INVENTION

All percentages are by weight of the personal-care composition, unless otherwise specified. All ratios are weight ratios, unless specifically stated otherwise. All numeric ranges are inclusive of narrower ranges; delineated upper and lower range limits are interchangeable to create further ranges not explicitly delineated. The number of significant digits conveys neither limitation on the indicated amounts nor on the accuracy of the measurements. All measurements are understood to be made at about 25° C. and at ambient conditions, where “ambient conditions” means conditions under about one atmosphere of pressure and at about 50% relative humidity.

“Mascara” and “mascara composition,” as used herein, mean a compound that is applied to eyelashes, eyelids, and/or eyebrows. Mascara compositions of the present invention may be formulated for topical application on mammalian keratinous tissue for use in skin-care, cosmetic, and hair-care products; non-limiting uses of which include antiperspirants, deodorants, lotions (e.g. hand lotion and body lotion), skin-care products (e.g., face and neck lotions, serums, sprays), sunless tanners, cosmetics (e.g., foundation, mascara, concealer, blush, lipstick, lip gloss, eyeliner, eye shadow, eyebrow pencil), hair dyes, after-shaves, razor moisturizing/lubricating strips, razor shave-gel bars, feminine-care products, oral-care products, and baby-care products. The methods of using any of the aforementioned compositions are also included within the meaning of mascara composition. “Keratinous tissue,” as used herein, refers to keratin-containing layers disposed as the outermost protective covering of mammals which includes, but is not limited to, skin, hair, and nails.

### Mascara Composition

The present invention relates to a method of coating eyelids and eyelashes comprising the step of applying to eyelashes, eyelids, or both, a mascara composition. The mascara composition remains intact on the eyelashes and/or eyelids of the wearer for greater than 24 hours, preferably greater than 36 hours, and even more preferably greater than about 48 hours. Solvent-based mascara-remover compositions are disclosed below because the mascara composition is generally not soluble in soap and water. The mascara composition comprises at least about 15%, preferably at least about 17%, and even more preferably at least about 20% to about 35%, by weight, of a film former selected from the group consisting of tall oil glyceride, pentaerythrityl rosinate, glyceryl rosinate, and the hydrogenated versions and mixtures thereof. The ratio of film former to volatile solvent is controlled such that the dried film consists of from about 30% to about 70% film former by weight. Other ingredients suitable for use in the compositions of the present invention are described and exemplified below.

A second essential component of the mascara composition is a colorant, which is described and exemplified below. Common colorants suitable for use in the mascaras compositions of the present invention include dyes and pigments. Preferably the colorant comprises at least about 0.1%, preferably at least about 0.5%, and even more preferably at least about 1.0% to about 14%, by weight, of the mascara composition. The mascara compositions can be a single coat application, a base coast of mascara and a top coat. Both embodiments, single coat and two coat, can be used with the solvent-based remover composition described below.

Similar to many “waterproof” mascara formulas, the inventive mascara is an anhydrous dispersion (not emulsion) comprised of colorant particles dispersed in a matrix of film former(s) and volatile solvent(s). Key compositional elements and features are outlined below.

Film Formers

An essential component of the invention is a film former from a class of rosinates or rosin esters. In a preferred embodiment, the mascara composition comprises film formers chosen from the group consisting of tall oil glycerides, pentaerythrityl rosinate, glyceryl rosinate, and mixtures thereof. These materials are derived from rosin. Rosin is a solid form of resin obtained from conifers, produced by heating liquid resin to vaporize the volatile liquid terpene components. It primarily consists of different resin acids, especially abietic acid. Rosin is typically obtained commercially either by distillation of volatile turpentine from oleoresin exuded from the wound of living pine trees to obtain gum rosin or the separation of tall oil, a byproduct of the wood pulp industry, to obtain tall oil rosin.

The carboxylic acid group of a rosin acid can be converted to an ester through a reaction with various alcohols. Esterification of rosin modifies the softening point, adhesiveness, cohesiveness, and melted viscosity of the material. The alcohols typically used to make rosin esters are methanol, tri-ethylene-glycol, glycerol, and pentaerythritol. Tall oil rosin is esterified with glycerol to form tall oil glycerides, a mixture of resin acids, rosin acids, and esters of glycerol. Tall oil glycerides are available from, for example, Arizona Chemical Co. Glyceryl Rosinate is the ester of rosin acids with glycerol. Pentaerythrityl rosinate, sometimes referred to as pentaerythritol rosinate, is the ester of rosin acids with pentaerythritol. It is used as a skin conditioning agent-emollient and viscosity increasing agent-nonaqueous in a few cosmetic formulations. Pentaterythrityl Rosinate is commercially available, for example, from Eastman.

Rosin's conjugated double bond makes it susceptible to oxidation, isomerization and other reactions. A common method to improve stability is to hydrogenate the rosin molecules. This is done by the addition of hydrogen to double bonds in the resin acid, typically catalyzed by nickel compounds or noble metals to form saturated ring structures. Hydrogenation greatly increases the molecule's resistance to oxidation and improves its color. The hydrogenation process can be controlled so that the rosin is either partially or fully hydrogenated. Hydrogenated rosins have specific advantages over non-hydrogenated rosin resins including lighter color, improved stability, and reduced skin sensitization. The hydrogenated versions of pentaerythrityl rosinate and glyceryl rosinate, pentaerythrityl hydrogenated rosinate (PHR) and glyceryl hydrogenated rosinate (GHR) may be used in the invention.

Film formers such as tall oil glycerides, pentaerythrityl rosinate, pentaerythrityl hydrogenated rosinate, glyceryl rosinate, and glyceryl hydrogenated rosinate are used in the present invention in higher concentration than previously used in mascara. The combination of film formers is from about 15% to about 35%, preferably from about 20% to about 30%, even more preferably from about 25% to about 30%. The ratio of film former (e.g., PHR, GHR, tall oil glycerides, and combinations thereof) to volatile carrier is controlled such that the dried film consists of from about 30% to about 70% film former by weight, preferably from about 40% to about 60%, and most preferably about 50%.

Carrier Solvents

The mascara composition comprises a carrier solvent to achieve delivery of the film formers to the eyelash or eyelid. In a preferred embodiment, the mascara composition comprises a volatile carrier which quickly volatilizes from the surface of the eyelashes or eyelid, leaving the desired components behind.

The volatile carrier comprises from about 10% to about 85%, preferably from about 15% to about 80%, and most preferably from about 20% to about 70% of the composition. The volatile carrier of the present invention is selected from the group consisting of volatile hydrocarbons, volatile silicones and mixtures thereof.

Hydrocarbon oils useful in the present invention include those having boiling points in the range of 60-260° C., more preferably hydrocarbon oils having from about C8 to about C20 chain lengths, most preferably C8 to C20 isoparaffins. Of these isopariffins most preferred are selected from the group consisting of isododecane, isohexadecane, isoeicosane, 2,2,4-trimethylpentane, 2,3-dimethylhexane and mixtures thereof. Most preferred is isododecane, available as for example Permethyl 99A from Presperse corresponding to the formula:

CH3 (CH2)10CH

Preferred volatile silicone fluids include cyclomethicones having 3, 4 and 5 membered ring structures corresponding to the formula:

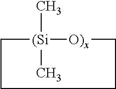

where X is from about 3 to about 6. Said volatile silicones include 244 Fluid, 344 Fluid and 245 Fluid, and 345 Fluid all from Dow Corning Corporation.

Colorants

Colorants suitable for use in the present invention include, but are not limited to, a dye, pigment, lake, effect pigment, and mixture thereof. Typical suitable colorants for eye-area cosmetics include any organic or inorganic pigment or colorant approved for use in eye-area cosmetics by CTFA and/or the FDA such as lakes, iron oxides, titanium dioxide, iron sulfides, ultramarines or other conventional pigments used in cosmetic formulations.

Examples of pigments include inorganic pigments such as chromium oxide greens, ultramarines, yellow iron oxide, brown iron oxide, red iron oxide, and titanium oxide; and organic pigments such as D&C Black No. 2, D&C Black No. 3, FD&C Red No. 40, D&C Green No. 5, FD&C Blue No. 1, and FD&C Yellow No. 5. Examples of lake dyes include various acid dyes which are laked with aluminum, calcium or barium.

The above pigments, dyes, and lakes are well known, commercially available materials, with their chemical structure being described, e.g., in 21 C. F. R. Part 74 (as revised Apr. 1, 1988) and in the CTFA Cosmetic Ingredient Handbook, (1988), published by the Cosmetics, Toiletry and Fragrances Association, Inc.

Encapsulated colorant microparticles having average diameters of 0.1 to 10 microns are acceptable for use in the compositions of the present invention, for example 0.1 to 5 and especially 0.1 to 1 microns. The encapsulated colorant microparticles may comprise 1 to 60% by weight of at least one colorant, for example 5-40% and especially 7 to 25% by weight.

Additionally, the microencapsulated colorants may provide a more vibrant color to products used around the eye area, including eyelashes. The primary colors are understood to mean red, yellow and blue. An additional feature of microparticles is the elimination of milling or grinding often encountered with non-encapsulated colorants. Said colorants are preferably organic.

Colorants that are surface modified with a hydrophobic coating are acceptable for use in the compositions of the present invention, for example Triethoxycaprylsilane. Hydrophobically coating of colorants may increase their dispensability in the non-polar solvent and increase their resistance to being washed off during exposure to showering and facial cleansing.

The colorant for use herein is preferably selected from the following exemplary list of currently available colorants approved by the FDA for use around the eyes. Other colorants may also be used as they are developed and determined safe. Colorants for use in the present invention can be selected from the group consisting of annatto, caramel, carmine, β-carotene, bismuth oxychloride, iron oxides, ferric ammonium ferrocyanide, ferric ferrocyanide, chromium hydroxide green, chromium oxide greens, guanine, mica, titanium dioxide, aluminum powder, bronze powder, copper powder, ultramarines, manganese violet, zinc oxide, D&C Black No. 2, D&C Black No. 3, FD&C Blue No. 1, D&C Green No. 5, FD&C Red No. 40, and FD&C Yellow No. 5.

Effect pigments useful in the present invention include, but are not limited to, pearls, nacres, and the effect pigments described in US2009/0220557 to Pfaff and U.S. Pat. No. 6,875,264 to Zimmerman.

The mascara composition according to the invention comprises from about 0.1 to about 70% by weight, for example from about 0.5 to about 50% by weight, and especially from about 0.5 to about 35% by weight based on the total weight of the composition, of a colorant. Colorants having average diameters of 0.1 to 10 microns are acceptable for use in the compositions of the present invention, for example 0.1 to 5 and especially 0.1 to 1 microns. It is preferable that the diameter of the colorant is smaller than the thickness of the mascara composition dried-down film. The preferred diameter of the colorant is less than 1 micron. The small size of the colorants allows them to be fully encased in the dried film. In certain embodiments, if effect pigments are used, average diameters in excess of 30 microns are acceptable for use.

Thickeners

The mascara composition can be thickened or structured with colloidal particles and/or waxes. Thickeners for use in the present invention can be selected from the group consisting of waxes such as carnauba wax, candellila wax, beeswax, and polyethylene wax; particles such as disteardimonium hectorite, kaolin, silica, and magnesium carbonate; polymers; viscous hydrocarbons; and combinations thereof. Waxes are selected to maintain the film durability of the mascara composition. The mascara composition according to the present invention comprises from about 3% to about 15% wax, preferably from about 4% to about 12% wax, most preferably from about 5% to about 10% wax. In one embodiment, the mascara composition comprises greater than about 10% wax, more preferably from about 10% to about 15% wax. Disteardimonium hectorite is the preferred thickener to build structure/viscosity. This enables proper spreading/deposition across lashes, and ensures adequate stability/suspension of colorant particles in dispersion over time. It is preferable that the diameter of the disteardimonium hectorite is smaller than the thickness of the mascara composition dried-down film. The preferred diameter of the disteardimonium hectorite is less than 10 microns. The mascara composition according to the present invention comprises from about 1% to about 25% disteardimonium hectorite, preferably from about 2% to about 20% disteardimonium hectorite, most preferably from about 3% to about 15% disteardimonium hectorite.

### Mascara Top Coat

A second composition may be placed over the mascara compositions of the present invention or over any commercially available mascara. These second, overlying compositions are referred to herein as a top coat. The preferred topcoat composition is anhydrous and designed to mitigate the tackiness of the base layer or basecoat as well as extend the wear of the product.

An essential component of the top coat is a film forming polymer. One example of a film forming polymer suitable for use in the present invention is an organosiloxane resin. The film forming polymers can also be incorporated into the mascara composition described above. The resin may comprise combinations of R3SiO1/2 “M” units, R2SiO “D” units, RSiO3/2 “T” units, SiO2 “Q” units in ratios to each other that satisfy the relationship RnSiO(4-n)/2 where n is a value between 1.0 and 1.50 and R is a methyl group. Up to 5% of silanol or alkoxy functionality may also be present in the resin structure as a result of processing. The organosiloxane resins have a number molecular weight average range of from about 1,000 g/mole to about 10,000 g/mole. The resin is soluble in organic solvents such as toluene, xylene, isoparaffins, and cyclosiloxanes or the volatile carrier, indicating that the resin is not sufficiently crosslinked such that the resin is insoluble in the volatile carrier. Particularly preferred are resins comprising repeating monofunctional or R3SiO1/2 “M” units and the quadrafunctional or SiO2 “Q” units, otherwise known as “MQ” resins as disclosed in U.S. Pat. No. 5,330,747, Krzysik, issued Jul. 19, 1994, incorporated herein by reference. In the present invention the ratio of the “M” to “Q” functional units is about 0.6 to about 2.0, more preferably about 0.6 to about 0.9, most preferably about 0.7. Examples of organosiloxane resins commercially available are Wacker 803 and 804 available from Wacker Silicones Corporation of Adrian Michigan, and G.E. SR1000 from the General Electric Company.

Organosiloxane resins are used in the present invention at levels from about 10% to about 95%, preferably from about 55% to about 80%, and most preferably 60% to about 70% of the total amount of top coat composition. In one embodiment of this invention the top coat comprises less than about 3.0%, preferably less than about 1.0%, even more preferably less than 0.1%, by weight, of colorants.

In a preferred embodiment, isododecane is used as a volatile solvent and trimethylsiloxysilicate (MQ Resin) and dimethicone are used as film-forming agents. These ingredients were selected because in combination they form flexible and non-tacky films.

### Mascara Remover

As discussed above, high concentrations of PHR and tall oils substantially reduce the solubility of the mascara in soap and water. Prior mascara formulations kept the concentration of these two components low to insure the mascara could be washed off in soap and water. Accordingly, in one aspect of this invention a solvent-based mascara-remover composition is provided. The mascara remover comprises at least about 40%, preferably at least about 50%, and even more preferably at least about 55% of a solvent selected from the group consisting of mineral oil, petroleum jelly, isododecane, silicones, other hydrocarbon solvents and mixtures thereof. The solvent can comprise up to about 100% of the remover composition.

### Optional Ingredients

The mascara, top coat, and mascara remover described above may contain other optional ingredients. Because there is some overlap between the mascara, top coat, and remover compositions described above and the optional ingredients which may be included in them, the optional ingredients are described together below.

Actives

The compositions of the present invention may comprise a safe and effective amount of a biological, chemical, nutraceutical, or pharmaceutical active, or a combination thereof. Biological actives may include prostaglandins, antimicrobials, antibacterials, biocides, preservatives, proteins, amino acids, peptides, hormones, growth factors, enzymes (e.g., glutathione sulphydryl oxidase, transglutaminase), therapeutics, oligonucleotides, genetic materials (e.g., DNA, RNA), and combinations thereof. Chemical actives may include dyes, surfactants, sensates, hair conditioners, hair dyes, hair growth agents, hair removers, hair growth inhibitors, hair styling gels, and combinations thereof. Nutraceutical actives may include proteins, preservatives, vitamins, food-additive materials, and combinations thereof. Pharmaceutical actives may include antibiotics, drugs, hair growth agents, hair removers, hair growth inhibitors, and combinations thereof.

Oil Soluble or Oil Dispersible Additives

The choice of oil-soluble or dispersible additive and the amount present according to the invention will depend on the intended use of the composition and the effectiveness of the compound. In mascara, top coat and remover compositions, the oil-soluble or dispersible additive chosen is acceptable for skin and eye contact, as is well known to the skilled formulator. Suitable oil-soluble or dispersible additives are incorporated at levels generally between 1 and 20% by weight based on the weight of the matrix bead (equivalent to 90 to 300% on weight of the colorant). Preferably 5 to 15% by weight of the oil-soluble or dispersible additive is employed.

The oil-soluble or dispersible additive may include fatty alcohols such as GUERBET alcohols based on fatty alcohols having from 6 to 30, preferably from 10 to 20 carbon atoms including lauryl alcohol, cetyl alcohol, stearyl alcohol, cetearyl alcohol, oleyl alcohol, benzoates of C₁₂-C₁₅ alcohols, acetylated lanolin alcohol, etc. Especially suitable is stearyl alcohol.

The oil-soluble or dispersible additive may include fatty acids such as Linear fatty acids of C₆-C₂₄, branched C₆-C₁₃carboxylic acids, hydroxycarboxylic acids, caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselinic acid, linoleic acid, linolenic acid, elaeostearic acid, arachidic acid, gadoleic acid, behenic acid and erucic acid and technical-grade mixtures thereof (obtained, for example, in the pressure removal of natural fats and oils, in the reduction of aldehydes from Roelen's oxosynthesis or in the dimerization of unsaturated fatty acids).

Further components that can be used are dicarboxylic acids of C₂-C₁₂, such as adipic acid, succinic acid, and maleic acid. Aromatic carboxylic acids, saturated and/or unsaturated, especially benzoic acid, can be used.

Additional components that can be used as the oil soluble or dispersible additive include carboxylic acid salts: for example the salts of C₈-C₂₄, preferably C₁₄-C₂₀ saturated or unsaturated fatty acids, C₈-C₂₂ primary or secondary alkyl sulfonates, alkyl glycerol sulfonates, the sulfonated polycarboxylic acids described in published British Patent 1,082,179, paraffin sulfonates, N-acyl, N′-alkyl taurates, alkyl phosphates, isethionates, alkyl succinamates, alkyl sulphosuccinates, monoesters or diesters of sulfosuccinates, N-acyl sarcosinates, alkyl glycoside sulfates, polyethoxycarboxylates, the cation being an alkali metal (sodium, potassium, lithium), an unsubstituted or substituted ammonium residue (methyl, dimethyl, trimethyl, tetramethyl ammonium, dimethyl piperidinium, etc.) or a derivative of an alkanol amine (monoethanol amine, diethanol amine, triethanol amine, etc.); alkaline soaps of sodium, potassium and ammonium; metallic soaps of calcium or magnesium; organic basis soaps such as lauric, palmitic, stearic and oleic acid, etc., alkyl phosphates or phosphoric acid esters: acid phosphate, diethanolamine phosphate, potassium cetyl phosphate.

Silicones and Siloxanes

Silicones or siloxanes (organosubstituted polysiloxanes) may be used herein. This includes, but is not limited to, dimethylpolysiloxanes, methylphenylpolysiloxanes, cyclic silicones, and also amino-, fatty acid-, alcohol-, polyether-, epoxy-, fluorine-, glycoside- and/or alkyl-modified silicone compounds, which at room temperature may be in either liquid or resinous form; linear polysiloxanes: dimethicones such as Dow Corning® 200 fluid, Mirasil® DM (Rhodia), dimethiconol; cyclic silicone fluids: cyclopentasiloxanes, volatiles such as Dow Corning® 345 fluid, Silbione® grade, Abil® grade; phenyltrimethicones; Dow Corning® 556 fluid. Also suitable are simethicones, which are mixtures of dimethicones having an average chain length of from 200 to 300 dimethylsiloxane units with hydrogenated silicates. A detailed survey by Todd et al. of suitable volatile silicones may be found in addition in Cosm. Toil. 91, 27 (1976). Especially suitable are ethoxylated propoxylated dimethicone (e.g. Dow Corning 5225C Formulation Aid) and aminopropyldimethicone (e.g. Tinocare SiAl from Ciba Specialty Chemicals).

Fluorinated or perfluorinated alcohols and acids may be used herein. This includes, but is not limited to, perfluordodecanoic acid, perfluordecanoic acid, perfluoro-tert-butyl alcohol, perfluoroadipic acid, 2-(perfluoroalkyl)ethanol (ZONYL® BA-L).

The oil-soluble or dispersible additive may be an anionic surfactant. Examples of such anionic surfactants include: alkyl ester sulfonates of the formula R₁₀₀—CH(SO₃M)-COOR₂₀₀, where R₁₀₀ is a C₈-C₂₀, preferably C₁₀-C₁₆ alkyl radical, R₂₀₀ is a C₁-C₁₆, preferably C₁-C₃ alkyl radical, and M is an alkaline cation (sodium, potassium, lithium), substituted or non-substituted ammonium (methyl, dimethyl, trimethyl, tetramethyl ammonium, dimethyl piperidinium, etc.) or a derivative of an alkanol amine (monoethanol amine, diethanol amine, triethanol amine, etc.); alkyl sulfates of the formula R₃₀₀OSO₃M, where R₃₀₀ is a C₅-C₂₄, preferably C₁₀-C₁₈ alkyl or hydroxyalkyl radical, and M is a hydrogen atom or a cation as defined above, and their ethyleneoxy (EO) and/or propyleneoxy (PO) derivatives, having on average 0.5 to 30, preferably 0.5 to 10 EO and/or PO units; alkyl amide sulfates of the formula R₄₀₀CONHR₅₀₀OSO₃M, where R₄₀₀ is a C₂-C₂₂, preferably C₆-C₂₀ alkyl radical, R₅₀₀ is a C₂-C₃ alkyl radical, and M is a hydrogen atom or a cation as defined above, and their ethyleneoxy (EO) and/or propyleneoxy (PO) derivatives, having on average 0.5 to 60 EO and/or PO units.

The oil-soluble or dispersible additive may be a non-ionic surfactant. Nonionic surfactants that may be used include the primary and secondary alcohol ethoxylates, especially the C₈-C₂₀ aliphatic alcohols ethoxylated with an average of from 1 to 20 moles of ethylene oxide per mole of alcohol, and more especially the C₁₀-C₁₅ primary and secondary aliphatic alcohols ethoxylated with an average of from 1 to 10 moles of ethylene oxide per mole of alcohol. Non-ethoxylated nonionic surfactants include alkylpolyglycosides, glycerol monoethers, and polyhydroxyamides (glucamides).

Some particular examples of such nonionic surfactants include: polyalkoxylenated alkyl phenols (i.e. polyethyleneoxy, polypropyleneoxy, polybutyleneoxy), the alkyl substituent of which has from 6 to 12 C atoms and contains from 5 to 25 alkoxylenated units; examples are TRITON X-45, X-114, X-100 and X-102 marketed by Rohm & Haas Co., and IGEPAL NP2 to NP17 made by Rhodia; C₈-C₂₂ polyalkoxylenated aliphatic alcohols containing 1 to 25 alkoxylenated (ethyleneoxy, propyleneoxy) units; examples include TERGITOL 15-S-9, TERGITOL 24-L-6 NMW marketed by Dow, NEODOL 45-9, NEODOL 23-65, NEODOL 45-7, and NEODOL 45-4 marketed by Shell Chemical Co., KYRO EOB marketed by The Procter & Gamble Co., SYNPERONIC A3 to A9 made by ICI, RHODASURF IT, DB and B made by Rhodia; the products resulting from the condensation of ethylene oxide or propylene oxide with propylene glycol and/or ethylene glycol, with a molecular weight in the order of 2,000 to 10,000, such as the PLURONIC products marketed by BASF; the products resulting from the condensation of ethylene oxide and/or propylene oxide with ethylene diamine, such as the TETRONIC products marketed by BASF; C₈-C₁₈ ethoxyl and/or propoxyl fatty acids containing 5 to 25 ethyleneoxy and/or propyleneoxy units; C₈-C₂₀ fatty acid amides containing 5 to 30 ethyleneoxy units; ethoxylated amines containing 5 to 30 ethyleneoxy units; alkoxylated amidoamines containing 1 to 50, preferably 1 to 25 and in particular 2 to 20 alkyleneoxy (preferably ethyleneoxy) units; amine oxides such as the oxides of alkyl C₁₀-C₁₈ dimethylamines, the oxides of alkoxy C₈-C₂₂ ethyl dihydroxy ethylamines; alkoxylated terpene hydrocarbons such as ethoxylated and/or propoxylated α- or β pinenes, containing 1 to 30 ethyleneoxy and/or propyleneoxy units; alkylpolyglycosides obtainable by condensation (for example by acid catalysis) of glucose with primary fatty alcohols (e.g. those in U.S. Pat. Nos. 3,598,865 and 4,565,647; and EP-A-132 043 and EP-A-132 046) having a C₄-C₂₀, preferably C₈-C₁₈ alkyl group and an average number of glucose units in the order of 0.5 to 3, preferably in the order of 1.1 to 1.8 per mole of alkylpolyglycoside (APG), particularly those having a C₈-C₁₄ alkyl group and on average 1.4 glucose units per mole, a C₁₂-C₁₄ alkyl group and on average 1.4 glucose units per mole, a C₈-C₁₄ alkyl group and on average 1.5 glucose units per mole or a C₈-C₁₀ alkyl group and on average 1.6 glucose units per mole, marketed under the names GLUCOPON 600 EC, GLUCOPON 600 CSUP, GLUCOPON 650 EC and GLUCOPON 225 CSUP respectively and made by Henkel.

Another class of suitable surfactants comprises certain mono-long chain-alkyl cationic surfactants. Cationic surfactants of this type include quaternary ammonium salts of the general formula R₁₀R₂₀R₃₀R₄₀N⁺X⁻ wherein the R groups are long or short hydrocarbon chains; typically alkyl, hydroxyalkyl or ethoxylated alkyl groups, and X is a counter-ion (for example, compounds in which R₁₀ is a C₈-C₂₂ alkyl group, preferably a C₈-C₁₀ or C₁₂-C₁₄ alkyl group, R₂₀ is a methyl group, and R₃₀ and R₄₀, which may be the same or different, are methyl or hydroxyethyl groups); and cationic esters (for example, choline esters).

Also useful are ethoxylated carboxylic acids or polyethylene glycol esters (PEG-n acylates), linear fatty alcohols having from 8 to 22 carbon atoms, products from 2 to 30 mol of ethylene oxide and/or from 0 to 5 mol propylene oxide with fatty acids having from 12 to 22 carbon atoms and with alkylphenols having from 8 to 15 carbon atoms in the alkyl group, fatty alcohol polyglycol ethers such as Laureth-n, Ceteareth-n, Steareth-n and Oleth-n, fatty acid polyglycol ethers such as PEG-n Stearate, PEG-n Oleate and PEG-n Cocoate; polyethoxylated or acrylated lanolin; monoglycerides and polyol esters; C₁₂-C₂₂ fatty acid mono- and di-esters of addition products of from 1 to 30 mol of ethylene oxide with polyols; fatty acid and polyglycerol esters such as monostearate glycerol, diisostearoyl polyglyceryl-3-diisostearates, polyglyceryl-3-diisostearates, triglyceryl diisostearates, polyglyceryl-2-sesquiisostearates or polyglyceryl dimerates. Mixtures of compounds from a plurality of these substance classes are also suitable. Fatty acid polyglycol esters such as monostearate diethylene glycol, fatty acid and polyethylene glycol esters; fatty acid and saccharose esters such as sucro esters, glycerol and saccharose esters such as sucro glycerides; sorbitol and sorbitan: sorbitan mono- and di-esters of saturated and unsaturated fatty acids having from 6 to 22 carbon atoms and ethylene oxide addition products; polysorbate-n series, sorbitan esters such as sesquiisostearate, sorbitan, PEG-(6)-isostearate sorbitan, PEG-(10)-laurate sorbitan, PEG-17-dioleate sorbitan; glucose derivatives: C₈-C₂₂ alkyl-mono and oligo-glycosides and ethoxylated analogues with glucose being preferred as the sugar component; 0/W emulsifiers such as Methyl Gluceth-20 sesquistearate, sorbitan stearate/sucrose cocoate, methyl glucose sesquistearate, cetearyl alcohol/cetearyl glucoside; also W/O emulsifiers such as methyl glucose dioleate/methyl glucose isostearate.

Oil-soluble or dispersible additives also include sulfates and sulfonated derivatives: e.g. dialkylsulfosuccinates (e.g. DOSS, dioctyl sulfosuccinate), alkyl lauryl sulfonate, linear sulfonated paraffins, sulfonated tetrapropylene sulfonate, sodium lauryl sulfates, ammonium and ethanolamine lauryl sulfates, lauryl ether sulfates, sodium laureth sulfates, acetyl isothionates, alkanolamide sulfates such as taurines, methyl taurines, and imidazole sulfates.

Oil-soluble or dispersible additives also include amine derivatives: amine salts, ethoxylated amines such as amine oxides, amines with chains containing a heterocycle such as alkyl imidazolines, pyridine derivatives, isoquinolines, cetyl pyridinium chloride, cetyl pyridinium bromide, quaternary ammonium compounds such as cetyltrimethylammonium bromide, and stearylalkonium salts; amide derivatives: alkanolamides such as acylamide DEA, ethoxylated amides, such as PEG-n acylamide, oxydeamide; polysiloxane/polyalkyl/polyether copolymers and derivatives: dimethicone, copolyols, silicone polyethylene oxide copolymers and silicone glycol copolymers; propoxylated or POE-n ethers (Meroxapols), Polaxamers or poly(oxyethylene)ₘ-block-poly(oxypropylene)ₙ-block(oxyethylene) copolymers; zwitterionic surfactants that carry at least one quaternary ammonium group and at least one carboxylate and/or sulfonate group in the molecule, zwitterionic surfactants that are especially suitable include the so-called betaines, such as N-alkyl-N,N-dimethylammonium glycinates, for example cocoalkyldimethylammonium glycinate, N-acylaminopropyl-N,N-dimethylammonium glycinates, for example cocoacylaminopropyldimethylammonium glycinate, and 2-alkyl-3-carboxymethyl-3-hydroxyethylimidazolines each having from 8 to 18 carbon atoms in the alkyl or acyl group and also cocoacylaminoethylhydroxyethyl-carboxy-methylglycinate, N-alkylbetaines and N-alkylaminobetaines; alkylimidazolines, alkylopeptides and lipoaminoacids; self-emulsifying bases (see K. F. DePolo—A Short Textbook Of Cosmetology, Chapter 8, Table 8-7, p 250-251); non-ionic bases such as PEG-6 Beeswax (and) PEG-6 stearate (and) polyglyceryl-2-isostearate [Apifac], Glyceryl stearate (and) PEG-100 stearate, [Arlacel 165], PEG-5 Glyceryl stearate [Arlatone 983 S], sorbitan oleate (and) polyglyceryl-3-ricinoleate [Arlacel 1689], sorbitan stearate and sucrose cocoate [Arlatone 2121], glyceryl stearate and laureth-23 [Cerasynth 945], cetearyl alcohol and Ceteth-20 [Cetomacrogol Wax], cetearyl alcohol and Polysorbate 60 and PEG-150 and stearate-20 [Polawax GP 200, Polawax NF], cetearyl alcohol and cetearyl polyglucoside [Emulgade PL 1618], cetearyl alcohol and Ceteareth-20 [Emulgade 1000NI, Cosmowax], cetearyl alcohol and PEG-40 castor oil [Emulgade F Special], cetearyl alcohol and PEG-40 castor oil and sodium cetearyl sulfate [Emulgade F], stearyl alcohol and Steareth-7 and Steareth-10 [Emulgator E 2155], cetearyl Alcohol and Steareth-7 and Steareth-10 [Emulsifying wax U.S.N.F], glyceryl stearate and PEG-75 stearate [Gelot 64], propylene glycol ceteth-3 acetate [Hetester PCS], propylene glycol isoceth-3 acetate [Hetester PHA], cetearyl alcohol and Ceteth-12 and Oleth-12 [Lanbritol Wax N 21], PEG-6 stearate and PEG-32 stearate [Tefose 1500], PEG-6 stearate and Ceteth-20 and Steareth-20 [Tefose 2000], PEG-6 Stearate and ceteth-20 and Glyceryl Stearate and steareth-20 [Tefose 2561], glyceryl stearate and Ceteareth-20 [Teginacid H, C, X]; anionic alkaline bases such as PEG-2 stearate SE, glyceryl stearate SE [Monelgine, Cutina KD] and propylene glycol stearate [Tegin P]; anionic acid bases such as cetearyl alcohol and sodium cetearyl sulfate [Lanette N, Cutina LE, Crodacol GP], cetearyl alcohol and sodium lauryl sulfate [Lanette W], Trilaneth-4 phosphate and glycol stearate and PEG-2 stearate [Sedefos 75], glyceryl stearate and sodium lauryl sulfate [Teginacid Special]; and cationic acid bases such as cetearyl alcohol and cetrimonium bromide.

Other useful oil-soluble or dispersible additives comprise mild surfactants, super-fatting agents, consistency regulators, additional thickeners, polymers, stabilizers, biologically active ingredients, deodorizing active ingredients, anti-dandruff agents, film formers, swelling agents, UV light-protective factors, antioxidants, preservatives, insect repellents, solubilizers, colorants, bacteria-inhibiting agents, hair conditioning agents, vitamins, and the like.

### Packaging

The mascara composition of the present invention may be packaged alone or in combination with a basecoat, topcoat, remover, eyeliner, eyebrow product, or combination thereof. In one embodiment, a mascara kit comprises a double-ended package, wherein the first end contains a mascara composition of the present invention and wherein the second end contains a mascara-remover composition of the present invention. In another embodiment, a mascara kit comprises a double-ended package, wherein the first end contains a mascara composition of the present invention and wherein the second end contains a topcoat composition.

A mascara, basecoat, topcoat, eyeliner, or eyebrow-product package (or complementary advertising) of the present invention may comprise indicia which suggests the long-wearing nature of the mascara composition. For instance, the indicia may comprise a certain number of hours the product may last, for example, 24 hours, 36 hours, or 48 hours. Or, the indicia may comprise a clock graphic. The indicia may be in a form selected from the group consisting of a label, a graphic, a three-dimensional shape, and combinations thereof. In one embodiment, the package comprises an hourglass indicia; the hourglass indicia may signal to the consumer that the product within is long-lasting. In a preferred embodiment, a mascara kit comprises a double-ended package, wherein the first end contains a mascara composition of the present invention and wherein the second end contains a mascara-remover composition of the present invention, and wherein the double-ended package comprises an indicia suggesting long-wear. Most preferably, the indicia comprises an hourglass.

### Uses

In addition to the conventional mascara uses (e.g., coloring the lashes), the mascara composition or topcoat of the present invention may be used for additional functions. For example, the mascara described herein may be used as an eyeliner, an eyebrow pencil, an eyebrow styling gel, an eyebrow highlighter, a basecoat, a primer, an active-delivery system, etc. The mascara composition described may also be used to attach adornments to the face or specifically, around the eye area. For instance, the mascara composition of the present invention may be used to attach false lashes, glitter, rhinestones, feathers, jewelry, etc to the face or eye area. Furthermore, the mascara composition of the present invention may be modified to create semi-permanent face makeup, such as foundation, concealer, blush, lipstick, etc, or to create a body concealer (e.g., to cover tattoos, blemishes, veins, or bruises on various parts of the body, such as the legs or back). One of ordinary skill in the art will recognize that not all of these forms require the presence of a colorant, e.g., a composition to attach adornments or a brow styling gel may be clear.

### Test Methods

In the Example section below, mascara compositions of the present invention are tested against current mascara compositions (products that are or were recently offered for sale to the public). Two test methods are used: the Flexibility Test and the Rub Test.

Flexibility Test Method

Flexibility is measured by the latex stretch test. This test predicts the ability of the color film (that is the mascara composition) to resist flaking and peeling after application. The film failure by flaking, peeling. and other methods is generally caused by movement of the skin, eyelids and eyelashes during normal activities. Failure can also occur by physical abrasion, such as rubbing the eyes or washing the face. The latex stretch test method is as follows:

Equipment:
1. Ansell Edmont Industrial technicians unlined gloves (12″ length, 17 mil) USDA Accepted #390, Size 9;
2. A disposable lip brush such as those available from La Femme Cosmetics, Inc. of L.A.
3. Analytical balance (4 decimal places);
4. Ruler; and
5. An apparatus as described and illustrated in FIG. 2 of U.S. Pat. No. 6,071,503. Said apparatus can be constructed from Lucite sheet and rod stock wherein posts 2a are approximately 11 inches apart.
6. Butler Gum Technique Full Size Head Toothbrush, Soft Bristles

Procedure:

(1) Cut a 1 inch wide band from the wrist area of the glove, avoiding the ribbing and thumb.

(2) Mark off a 1×1 inch block in the center of the band, avoiding the embossed number.

(3) Using a disposable lip brush, evenly apply 40 mg+/−2 mg over the 1×1 inch area of the band as marked in step (2).

(4) Allow the sample on the latex band from step (6) to sit at ambient room conditions for 10 minutes. Then place the sample in a 50° C. oven for 1 hour. After an hour, remove the sample from the oven and allow the sample to sit at ambient room conditions for 10 minutes.

(5) Weigh and record the combined weight of the latex band A and the applied cosmetic film; hereinafter referred to as A.

(6) Stretch the band to a length of 11 inches 5 times, allowing the band to return to its approximate original shape after each stretch. On the 5^{th} stretch, slip the band over the posts (2a) of apparatus (1)

(7) Rub the film surface with 5 strokes using the Gum toothbrush.

(8) Carefully remove the latex band from the posts (2a) allowing it to returns to its approximate original shape.

(9) Record the weight of the latex band (with the remaining cosmetic); herein referred to as B.

(10) Calculate the weight loss of the cosmetic film using the following equation:

Weight Loss (WL)=A−B

Steps (1) through (10) are repeated three times for each cosmetic formula tested. The average of the three WL values is determined; herein referred to as Average Weight Loss; or AWL. A Low AWL value corresponds to flexible films having desirable adhesive and cohesive balance of the film. The AWL for compositions of the present invention is 0.003 g and less, most preferably 0.

Rub Test Method

This test predicts the ability of a cosmetic film to resist color transfer to objects contacting the eyelash. Such objects may include hands, clothing, handkerchiefs or tissues, napkins, wash clothes, towels and the like.
Equipment: 10″×5½″ Leneta Form 2A Opacity Charts
   0.006″ draw down bar
   Draw down board
   Kimberly-Clark “WypAll” Paper towels
   2½″ diameter 2 kg weight
   1½″ diameter arch punch
   Scissors
   50° C. Oven
   Double sided tape
   Datacolor Microflash 200d

### Procedure:

1. Drawdown Preparation:
   a. Place a Leneta card on a drawdown board, Black/White side up. Label the top right of the card with sufficient sample & solvent identification. Take care to touch the card as little as possible since skin oils can affect the film thickness of product on the card.
   b. Apply 1-2 ml of product in a line across the top of the card, and use a 0.006″ drawdown bar to draw a film down the entire length of the card.
   c. Repeat steps 1a & 1b for all products to be tested.
   d. Once all draw downs have been completed, place them in a 50° C. oven for one hour. Take care not to mar the film surfaces.
   e. After an hour, remove the samples from the oven and allow them to equilibrate to ambient room temperature.
2. Sample Abrasion:
   a. Use an arch punch to punch out 1½″ diameter “abrasion substrate” disks from “WypAll” paper towels.
   b. Completely and evenly, (do not overlap) apply strips of double sided tape to the bottom of a 2 kg weight. Cut away excess tape that extends beyond the perimeter of the weight bottom. Place and adhere a single WypAll disk (1 ply thickness) to the sticky bottom of the weight. This will be used to abrade the film and see how much of it is removed.
   c. Place the disk/weight in the center of the first 3″×3″ testing area. Twist the disk through two full revolutions in ⅛ revolution increments. Carefully remove the weight (up and away from the drawdown), and remove the abrading disk from the bottom of the weight. (Only change the double sided tape when a disk will no longer firmly adhere to the bottom of the weight.)
   d. Perform 2a, 2b, & 2c for all subsequent Leneta card drawdowns.
3. Color Measurement
   a. Turn on the Microflash by flipping the toggle switch located on the back panel.
   b. Press the ‘Menu’ key on the front of the microflash until the word “Set-up” appears.
   c. Click the button directly below the word “set-up” to select the set-up menu.
   d. Use the arrow keys to pick the calibration program.
   e. Set the ‘hand-held head’ to Specular Included. (Using the toggles on the head, confirm that “white” (vs black) is showing through port.)
   f. The calibration program prompts you to place the white tile under the microflash head and press the trigger.
   g. The prompt that instructs you to measure the Black Trap. Place the black trap under the head and squeeze the trigger.
   h. The calibration is now complete.
   i. Using the toggles on the back of the Microflash's hand-held head, set the Specular to “Excluded”. (Confirm that “black” is showing through the port.)
   j. Select ‘Illuminant’ from the Main Menu. Use the D65/10 setting.
   k. Select Display from the main menu. Select CIE LCH Data and choose add.
   l. To take measurements, press the large Menu Key on the front of the Microflash.
   m. Place the Microflash over an unused “WypAll” disc and take a reading by squeezing the trigger. Then place the Microflash over a “WypAll” disc that has abraded a product film and take a reading by squeezing the trigger.
   n. Record the Delta L value (DL)
   o. Wipe/clean measuring surface in between samples.

Steps (1) through (3) are repeated three times for each cosmetic formula tested. The average of the three DL values is determined; herein referred to as Average Delta L Loss or Avg. DL. Low Avg. DL values correspond to films having greater abrasion resistance. The Avg. DL for compositions of the present invention are 4.00 and less, preferably about 3.00 and less, most preferably about 2.00 or less.

### MASCARA COMPOSITION EXAMPLES

### Example 1

A mascara composition according to the present invention is made having the composition of Table 1 according to the method given directly below Table 1.

TABLE 1
Mascara Composition

| Phase | Material | Weight % |
|---|---|---|
| A | Tall Oil Glycerides | 8.000 |
| A | Pentaerythrityl Hydrogenated Rosinate | 12.00 |
| A | Trihydroxystearin | 2.500 |
| A | Propylparaben | 0.100 |
| A | BHA | 0.100 |
| A | Phenoxyethanol 99% | 0.800 |
| A | Petroleum Distillates | 57.50 |
| B | Disteardimonium Hectorite | 5.000 |
| C | Propylene Carbonate | 2.500 |
| D | Black Iron Oxide | 5.500 |
| E | Encapsulated Blue Pigment | 6.000 |
| TOTAL | | 100.000 |

Phase A ingredients are melted and mixed together with low shear mixing. Phase B is gradually added to the Phase A and then dispersed with high shear mixing. Phase C is then added and mixed in with high shear mixing. Phase D is then added and dispersed with high shear mixing. The batch is cooled to ambient conditions and then Phase E is added and mixed in.

### Example 2

Additional mascara compositions according to the present invention, Nos. 1-4, are prepared and have the compositions given below in Table 2A.

TABLE 2A
Inventive Mascara Compositions

| Raw Material | Supplier/Trade | 1 | 2 | 3 | 4 |
|---|---|---|---|---|---|
| Description | Name | % W | % W | % W | % W |
| Isododecane | Presperse | 46.00% | 46.00% | 54.00% | 56.00% |
| | Permthyl 99A | | | | |
| Pentaerythrityl | Eastman Foral | 12.50% | 12.50% | 8.50% | 7.50% |
| Hydrogenated | 105-E | | | | |
| Rosinate | | | | | |
| Tall Oil | Arizona | 12.50% | 12.50% | 8.50% | 7.50% |
| | Chemical | | | | |
| | Sylvagum RE 85K | | | | |
| Disteardimonium | Elementis | 14.00% | 14.00% | 14.00% | 14.00% |
| Hectorite | Bentone 38V CG | | | | |
| Propylene | Huntsman Jeffsol | 4.50% | 4.50% | 4.50% | 4.50% |
| Carbonate | | | | | |
| 1,2 Hexanediol, | Symrise Symdiol | 1.00% | 1.00% | 1.00% | 1.00% |
| Caprylyl Glycol | 68 | | | | |
| Silicone Treated | Sensient Unipure | 0 | 9.50% | 9.50% | 9.50% |
| Black Iron Oxide | Black LC 989 | | | | |
| (Jet Milled) | EM AS | | | | |
| Untreated | Sensient Unipure | 9.50% | 0 | 0 | 0 |
| Black Iron Oxide | Black LC 989 | | | | |
| (Jet Milled) | EM | | | | |

| | | | | |
|---|---|---|---|---|
| TOTAL | 100.00% | 100.00% | 100.00% | 100.00% |

Numerous current market mascaras, products recently available for sale to the general public, are purchased and tested. These products all advertise better or longer wear characteristics than standard mascara products. Regardless, the comparative mascara products that are tested are given in Table 2B below.

TABLE 2B
Comparative Mascara Products

| | | Comparative |
|---|---|---|
| Manufacturer | Mascara Name | Product No. |
| Cover Girl ® | Marathon ® Water Proof | 5 |
| Cover Girl ® | Lash Exact ® | 6 |
| Avon ®* | Perfect Wear ® (16 Hour) | 7 |
| Sephora ® | Tarte 4 day Stay Lash Stain ® | 8 |
| Maybelline ® | Great Lash ® Water Proof | 9 |

*This product is no longer available for sale to consumers; all other comparative products are available to consumers.

The two test methods are described above and the products tested and the results of the tests are given below in Table 2C.

TABLE 2C
Test Results

| | |
|---|---|
| Flexibility Test | Rub Test |

| Inventive Composition No. | | | | |
|---|---|---|---|---|
| 1 | 0.0 | mg | 2.13 | ΔL |
| 2 | 0.0 | mg | 0.78 | ΔL |
| 3 | Not Tested | | 1.70 | ΔL |
| 4 | Not Tested | | 0.29 | ΔL |
| Inventive Average | 0.0 | mg | 1.23 | ΔL |
| Comparative Product No. | | | | |
| 5 | 2.0 | mg | 4.64 | ΔL |
| 6 | 4.0 | mg | 4.76 | ΔL |
| 7 | 12.0 | mg | 12.10 | ΔL |
| 8 | 15.0 | mg | 11.66 | ΔL |
| 9 | 6.0 | mg | 10.85 | ΔL |
| Comparative Average | 7.8 | mg | 8.80 | ΔL |

As is evident, the mascara compositions of this invention (Nos. 1-4) performed better individually and better on average than did any of the current market products (Nos. 5-9).

### Example 3

Another mascara composition is made according to the method below, having the composition according to Table 3.

TABLE 3
Inventive Mascara Composition

| | | 10 | 11 | 12 |
|---|---|---|---|---|
| Phase | Material | Weight % | Weight % | Weight % |
| A | Tall Oil Glycerides | 12.000 | 18.000 | 22.000 |
| A | Pentaerythrityl Hydrogenated | 13.000 | 0.000 | 10.000 |
| | Rosinate | | | |
| A | Glyceryl Monostearate | 5.750 | 6.750 | 4.750 |
| A | Black Iron Oxide | 7.750 | 8.750 | 6.750 |
| A | Disteardimonium Hectorite | 2.750 | 2.750 | 2.750 |
| A | Stearic Acid | 2.750 | 2.750 | 2.250 |
| A | Triethanolamine | 1.750 | 1.750 | 1.250 |
| A | Polyvinyl Alcohol | 2.000 | 2.000 | 2.000 |
| A | Propylene Carbonate | 0.750 | 0.750 | 0.250 |
| A | Lecithin | 1.750 | 1.750 | 1.250 |
| A | Oleic Acid 80% | 1.500 | 1.500 | 1.500 |
| B | Acrylates Copolymer | 5.170 | 5.170 | 2.170 |
| B | Isododecane | 15.200 | 18.200 | 10.200 |
| B | Simethicone Emulsion 30% | 0.200 | 0.200 | 0.200 |
| C | Xanthan Gum | 0.600 | 0.600 | 0.600 |
| C | Propylene Glycol | 3.500 | 3.500 | 3.500 |
| D | Ammonium Acrylates | 17.800 | 17.800 | 12.800 |
| | Copolymer | | | |
| E | Ethyl Alcohol SD 40-B | 1.000 | 1.000 | 1.000 |
| E | Benzyl Alcohol | 0.650 | 0.650 | 0.650 |
| E | Panthenol | 0.280 | 0.280 | 0.280 |
| E | Phenoxyethanol | 0.250 | 0.250 | 0.250 |
| E | Methylparaben | 0.200 | 0.200 | 0.200 |
| E | Ethylparaben | 0.200 | 0.200 | 0.200 |
| E | Propylparaben | 0.100 | 0.100 | 0.100 |
| E | Trisodium EDTA | 0.100 | 0.100 | 0.100 |
| F | Encapsulated Blue Pigment | 3.00 | 3.00 | 3.00 |
| | TOTAL | 100.000 | 100.000 | 100.000 |

Phase A is heated to allow the pigment to be dispersed with a Cowles Blade mixer. Phase B materials are stirred together at ambient conditions, and Phase C materials are stirred together at ambient conditions and then it is added to Phase B (to gel Phase B), and the mixture is stirred and then heated to about 85° C. Phase A and Phases B/C are mixed together to create an oil (wax) in water emulsion. The mixture is stirred for 15 minutes and then is cooled gradually to room temperature. During the cool down, Phases D and E are added to the mixture and stirred in below 60° C. Phase F is added to and mixed with the mascara once the mascara has cooled down to about 25-50° C.

### Example 4

Table 4 lists the composition for an exemplary top coat for use over the mascara compositions of the present invention.

TABLE 4
Top Coat Composition

| Raw Material | | | |
|---|---|---|---|
| Description | Supplier/Trade Name | Function | % Wt/Wt |
| Isododecane | Presperse Permthyl 99A | Volatile | 49.995% |
| | | Solvent | |
| Trimethyl- | Momentive MQ Resin | Film Former | 38.025% |
| siloxysilicate | | | |
| Dimethicone | Momentive SE-30 Gum | Film Former | 10.980% |
| 1,2 Hexanediol, | Symrise Symdiol 68 | Preservative | 1.000% |
| Caprylyl Glycol | | | |

| | |
|---|---|
| TOTAL | 100.000% |

### Example 5

Table 5 lists exemplary mascara compositions for use as eyeliners and concealer.

TABLE 5

| Raw Material | Solid Eyeliner | Liquid Eyeliner | Concealer |
|---|---|---|---|
| Description | Weight % | Weight % | Weight % |
| Isododecane | 39.5 | 51 | 56.510 |
| 1,2 Hexanediol and | 1 | 1 | 1 |
| Caprylyl Glycol | | | |
| Tall Oil Glycerides | 15 | 15 | 12.5 |
| Pentaerythrityl | 15 | 15 | 12.5 |
| Hydrogenated Rosinate | | | |
| Black Iron Oxide & Silica | 3.922 | 10 | 0.270 |
| (Jet Milled) | | | |
| Red Iron Oxide & Silica | 2.748 | 0 | 0.580 |
| (Jet Milled) | | | |
| Yellow Iron Oxide & | 2.33 | 0 | 0.940 |
| Silica (Jet Milled) | | | |
| Titanium Oxide | 0 | 0 | 7.700 |
| Disteardimonium | 8 | 8 | 8 |
| Hectorite | | | |
| Polyethylene wax | 12.5 | 0 | 0 |
| TOTAL | 100 | 100 | 100 |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of coating eyelids or eyelashes comprising the step of applying to eyelashes, eyelids, or both, a mascara composition comprising:
a. from about 15% to about 50%, by weight, of a film former selected from the group consisting of tall oil glycerides, pentaerythrityl rosinate, glyceryl rosinate, and the hydrogenated versions and mixtures thereof;
b. from about 0.1% to about 14%, by weight, of a colorant; and
c. from about 3% to about 15%, by weight, of a wax.

2. The method of claim 1, wherein the mascara composition comprises from about 4% to about 12% by weight of a wax.

3. The method of claim 1, wherein the mascara composition comprises from about 5% to about 10% by weight of a wax.

4. The method of claim 1, wherein the composition scores less than 1.0 in a Flexibility Test.

5. The method of claim 1, wherein the composition scores less than 4.5 in a Rub Test.

6. The method of claim 1, wherein the composition scores less than 3.0 in a Rub Test.

7. The method of claim 1, wherein the composition scores less than 2.5 in a Rub Test.

8. The method of claim 1, wherein the composition remains on the eyelashes or eyelids for more than about 24 hours.

9. The method of claim 1, wherein the composition remains on the eyelashes or eyelids for more than about 36 hours.

10. The method of claim 1, wherein the composition remains on the eyelashes or eyelids for more than about 48 hours.

11. The method of claim 1, wherein the mascara composition comprises from about 17% to about 30%, by weight, of a component selected from the group consisting of tall oil glyceride, pentaerythrityl hydrogenated rosinate, and mixtures thereof.

12. The method of claim 1, further comprising the step of applying to the eyelash or eyelid a second layer comprising a clear polymer selected from the group consisting of organosiloxane resins and mixtures thereof.

13. The method of claim 12, wherein the second layer comprises no colorant.

14. The method of claim 1, wherein the mascara composition comprises from about 15% to about 50%, by weight, of one or more tall oils.

15. The method of claim 1, wherein the mascara composition comprises from about 1% to about 20%, by weight, of disteardimonium hectorite.

16. The method of claim 1, wherein the mascara composition comprises a volatile carrier, wherein the ratio of film former to volatile carrier is controlled such that the dried mascara composition film consists of from about 30% to about 70% film former by weight.

17. The method of claim 1, wherein the mascara composition is contained by a mascara package, wherein the mascara package comprises indicia suggesting long wear.

18. The method of claim 17, wherein the indicia comprises an hourglass.

19. A mascara kit comprising a double-ended package, wherein the first end contains a mascara composition according to claim 1 and the second end contains a mascara-remover composition.

20. The mascara kit of claim 19, wherein the package comprises an hourglass indicia suggesting long wear.
